(19) United States
(12) Patent Application Publication
(10) Pub. No.: US 2020/0054693 A1
(43) Pub. Date: Feb. 20, 2020

(54) Probiotic Solution

(71) Applicant: Scott Chamberlin, Los Angeles, CA (US)

(72) Inventor: Scott Chamberlin, Los Angeles, CA (US)

(21) Appl. No.: 16/104,549

(22) Filed: Aug. 17, 2018

(51) Int. Cl. A61K 35/747

(52) U.S. Cl. CPC A61K 35/747; A61K 2035/115

(57) ABSTRACT

Probiotic material to allow exposure of a baby born by non-natural childbirth, to bacterial flora that simulates that obtained during the natural birth process. A topical probiotic is described that is put on a baby when it is first born, and mimics the bacteria in the mother's vaginal canal. This topical probiotic re-creates the bacteria that the baby would have received had the baby been naturally born. In embodiments, a bacterial culture of the probiotic material includes at least Lactobacillus crispatus, Lactobacillus iners, Lactobacillus. jensenii, and Lactobacillus gasseri.

### BACKGROUND

During natural childbirth, a baby passes through the woman's vaginal canal. However, due to pregnancy complications, many babies are born through C-sections in which the baby does not pass through the vaginal canal.

### SUMMARY

The inventor believes that the exposure of the baby to bacterial flora during the natural birth process, provides important immunities to the baby that jump start the baby's immunities at the first moments of its life. These exposures inoculate the baby's immune system, in a way that non-natural childbirth babies do not receive.

Embodiments of the invention describe a topical probiotic that is put on a baby when it is first born, and mimics the bacteria in the mother's vaginal canal. This topical probiotic re-creates the bacteria that the baby would have received had the baby been naturally born.

### DETAILED DESCRIPTION

An embodiment describes a topical probiotic which includes bacteria that mimics that the bacterial spectrum in the vaginal canal of an expectant mother.

The probiotic is applied to the baby after a non-natural childbirth, to mimic what the baby would have received had that baby have been naturally born. The probiotic is intended to be used on babies who are born by non-natural childbirth such a C-section.

In an embodiment, a number of different forms of the probiotic are created, each form of the probiotic being created for a particular ethnic group and place where the mothers live. For example, individual probiotics may be created for North America, Europe, England, and Asia. Within each group, there can be probiotic mixtures for Asian ethnicity, Caucasian ethnicity, or black ethnicity, for example.

The probiotic for the particular location where the user lives, and for the ethnicity is then used on a baby who is born using non-natural childbirth. The vaginal biome of the mother at the moment of birth is simulated by the probiotic material.

The vaginally biome is characteristically dominated by 5 major groups that reflect vaginal bacterial community.

The most commonly observed species is Lactobacillus species. A typical concentration is Lactobacillus crispatus, 40%, Lactobacillus iners, 27%, Lactobacillus. jensenii, 13% and Lactobacillus gasseri, 9%.

Other bacteria, in the vaginal canal is not necessarily simulated, especially that bacterial species associated with bacterial vaginosis including Prevotella species, Clostridium species, Atopobium species, and Megasphaera Species. It is believed that these undesirable species are less likely to be located in pregnant women.

Other species, such as L amylovorous (3%) may also be present.

In an embodiment, a topical probiotic is created which includes the most commonly occurring biome materials, including at least Lactobacillus species, preferably including at least Lactobacillus. crispatus, Lactobacillus iners, Lactobacillus. jensenii, and Lactobacillus gasseri, 9% 0 this tropical topical probiotic is applied to a baby as soon after non-childbirth as possible. If possible, it is allowed to dry or otherwise left on the baby for 15 to 20 minutes.

In another embodiment, a tropical probiotic is used which has bacterial colonies which are tied in type to the location where the user lives and their ethnic background.

In another embodiment, the mother's vaginal canal is cultured at a time between 7 and 8 months of pregnancy. The culture is either grown or analyzed to determine the bacterial concentration, and a compounding pharmacy is used to create the probiotic cream which is used on the baby after birth.

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art. For example, other sizes of plenums and fans can be used.

Also, the inventor(s) intend that only those claims which use the words “means for” are intended to be interpreted under 35 USC 112, sixth paragraph. Moreover, no limitations from the specification are intended to be read into any claims, unless those limitations are expressly included in the claims.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A probiotic material, having a bacterial culture therein, including at least multiple different species of Lactobacillus.

2. The probiotic material as in claim 1, wherein the bacterial culture of the probiotic material includes at least Lactobacillus crispatus.

3. The probiotic material as in claim 1, wherein the bacterial culture of the probiotic material includes at least Lactobacillus crispatus, Lactobacillus iners, Lactobacillus. jensenii, and Lactobacillus gasseri.

4. The probiotic material as in claim 1, wherein the specific bacteria, and the specific amounts of the bacteria are set based on mother's ethnicity and the place where the mother lives.

4. A method of applying a probiotic material to a baby, comprising:
obtaining a probiotic material which includes at least multiple different species of Lactobacillus; and
applying the probiotic material to a baby after a non-natural childbirth.

5. The method as in claim 4, wherein a bacterial culture of the probiotic material includes at least Lactobacillus crispatus.

6. The method as in claim 4, wherein a bacterial culture of the probiotic material includes at least Lactobacillus crispatus, Lactobacillus iners, Lactobacillus. jensenii, and Lactobacillus gasseri.

7. The method as in claim 4, further comprising determining a mother's ethnicity, and setting specific bacteria in the bacterial culture, and specific amounts of the bacteria in the bacterial culture, based on mother's ethnicity and a place where the mother lives.
